(19) United States
(12) Patent Application Publication
(10) Pub. No.: US 2020/0368369 A1
(43) Pub. Date: Nov. 26, 2020

(54) COMPOSITION FOR ENDOGENOUS PRODUCTION OF CHECKPOINT PROTEIN PRECURSORS

(71) Applicant: Wyvern Pharmaceuticals Inc., Calary (CA)

(72) Inventors: Jill L. Thompson, Calgary (CA); Bradley G. Thompson, Calgary (CA)

(21) Appl. No.: 16/419,874

(22) Filed: May 22, 2019

(51) Int. Cl. A61K 48/00; C12N 15/86; A61K 35/30; A61K 35/15; A61K 35/28; A61K 35/17; A61K 35/52; A61K 35/55

(52) U.S. Cl. CPC A61K 48/0016; C12N 15/86; A61K 35/30; A61K 35/15; A61K 2035/122; A61K 35/17; A61K 35/52; A61K 35/55; A61K 35/28

(57) ABSTRACT

The present disclosure relates to one or more agents, therapies, treatments, and methods of use of the agents and/or therapies and/or treatments for upregulating production and/or functionality of one or more protein precursors of IDO-1, CTLA-4, PD-1, PD-L1, PD-L2 and INF-y. Embodiments of the present disclosure can be used as a therapy or a treatment for a subject that has a condition whereby the subject's immune system is or is likely to become, dysregulated and where the upregulation of these protein precursors may be of therapeutic benefit.

### TECHNICAL FIELD

The present disclosure generally relates to molecules that are precursors of checkpoint molecules within a subject. In particular, the present disclosure relates to compositions, and/or the production, of one or more of the precursor proteins of checkpoint molecules and/or regulatory molecules of a checkpoint molecule.

### BACKGROUND

The immune system has evolved to differentiate between self and foreign matter. A number of cascades of signaling molecules and immune cells are characterized by their ability to recognize foreign matter and to call upon the production and stimulation of effector cells of the immune system to kill, break down, consume, or sheath the foreign matter in order to protect a host.

It is known that under various conditions the immune system can become dysregulated. A dysregulated immune system can cause further damage to the host, thereby preventing healing. It may also result in a loss of homeostatic controls and/or a chronically stimulated immune system.

Immune checkpoint molecules, in particular checkpoint proteins (CPP), are known participants in the immune system's responses to foreign matter. CPP can be categorized as stimulatory or inhibitory. Many stimulatory CPP have been identified as participating, either directly or indirectly, in increasing a host's immune response or protecting cells from the host's immune response. Many inhibitory CPP have also been identified as functioning to participate, either directly or indirectly, in decreasing a host's immune response. For example, it is known that the exogenous addition of a single CPP to cell surfaces may have therapeutic benefit in conditions where the immune system is dysregulated.

Known approaches to the treatment of conditions whereby the immune system is dysregulated are the commercially available pharmaceutical products Abatacept and Belatacept. These products can act as exogenous mimics of inhibitory CPP. For example, Abatacept is composed of the fragment crystallizable (Fc) portion of an immunoglobulin G1 (IgG1) that is linked to an extracellular domain of cytotoxic T-lymphocyte-associated protein 4 (CTLA-4). Mechanistically, Abatacept and Belatacept interfere with antigen-presenting cells' ability to generate a co-stimulatory signal, which is necessary to activate T-cells. Abatacept is used to treat rheumatoid arthritis and juvenile idiopathic arthritis. Abatacept has also been shown to be efficacious in psoriasis (phase 1 study results). Belatacept is used in renal transplant patients. Study results indicate that Belatacept may be as efficacious as cyclosporine at decreasing the immune reaction. Belatacept has also been shown to be efficacious in rheumatoid arthritis (phase 2 study results).

### SUMMARY

Some embodiments of the present disclosure relate to a method of making an agent/target cell complex, the method comprising a step of administering a therapeutically effective amount of the agent to a subject, wherein the agent/target cell complex increases the subject's production and/or functionality of one or more precursor proteins of checkpoint protein(s) (PCPP) and/or regulatory molecules of a PCPP.

Some embodiments of the present disclosure relate to a method of making an agent/target cell complex, the method comprising a step of administering a sufficient amount of an agent to a target cell whereby the agent/target cell complex is formed, wherein the agent/target cell complex increases the production and/or functionality of one or more PCPP and/or regulatory molecules of a PCPP by said target cell.

Some embodiments of the present disclosure relate to a pharmaceutical agent that comprises an agent, a pharmaceutically acceptable carrier and/or an excipient. The agent may upregulate production or functionality of one or more of PCPP and/or a regulatory molecule that upregulates the production or functionality of one or more PCPP.

Some embodiments of the present disclosure relate to a method of treating a condition. The method comprises a step of administering to a subject a therapeutically effective amount of an agent that upregulates the production and/or functionality of one or more PCPP and/or regulatory molecules of PCPP.

Some embodiments of the present disclosure relate to a use of an agent for treating a condition, wherein the agent upregulates the production and/or functionality of one or more PCPP and/or regulatory molecules of a PCPP in a subject that receives the agent.

Some embodiments of the present disclosure relate to a method for upregulating the production and/or functionality of one or more of PCPP and/or regulatory molecules of a PCPP, the method comprising a step of making an agent/target cell complex.

An agent that preferentially induces endogenous production of one or more PCPP and/or regulatory molecules of a PCPP, for example by upregulating the presence of PCPP on the subject's cellular surfaces, may be more effective at masking the target cells from the immune system than an exogenously added single inhibitory CPP or single inhibitory CPP mimic.

Embodiments of the present disclosure relate to at least one approach for inducing endogenous production of one or more PCPP and/or regulatory molecules of a PCPP. A first approach utilizes gene vectors containing nucleotide sequences and/or genes for one or more inhibitory PCPP (CTLA-4, PD-1, PD-L1, PD-L2, IDO-1) and/or regulatory molecules of an inhibitory PCPP (such as gamma interferon, also referred to herein as INF-γ), which can be administered to subjects to increase the production and/or functionality of endogenous PCPP.

Without being bound by any particular theory, embodiments of the present disclosure may be useful for treating conditions wherein the subject's immune system is, or is likely to become, dysregulated. Embodiments of the present disclosure relate to upregulating the production of one or more PCPP and/or one or more regulatory molecules of a PCPP for use as a therapy or a treatment for a subject that has a condition whereby the subject's immune system is, or is likely to become, dysregulated.

### DETAILED DESCRIPTION

### Definitions

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “activity” is used interchangeably with the term “functionality” and both terms refer to the physiologic action of biomolecule.

As used herein, the term “agent” refers to a substance that, when administered to a subject, causes one or more chemical reactions and/or one or more physical reactions and/or or one or more physiological reactions and/or one or more immunological reactions in the subject.

As used herein, the term “ameliorate” refers to improve and/or to make better and/or to make more satisfactory.

As used herein, the term “biomolecule” refers to a carbohydrate, a protein, an amino acid sequence, a nucleic acid, a lipid, a primary metabolite, a secondary metabolite that is found within a subject. A biomolecule may be endogenous or exogenous to a subject.

As used herein, the term “cell” refers to a single cell as well as a plurality of cells or a population of the same cell type or different cell types. Administering an agent to a cell includes in vivo, in vitro and ex vivo administrations and/or combinations thereof

As used herein, the term “complex” refers to an association, either direct or indirect, between one or more particles of an agent and one or more target cells. This association results in a change in the metabolism of the target cell. As used herein, the phrase “change in metabolism” refers to an increase or a decrease in the one or more target cells' production of deoxyribonucleic acid (DNA), ribonucleic acid (RNA), one or more proteins, and/or any post-translational modifications of one or more proteins.

As used herein, the terms “dysregulation” and “dysregulated” refer to situations or conditions wherein homeostatic control systems have been disturbed and/or compromised so that one or more metabolic, physiologic and/or biochemical systems within a subject operate partially or entirely without said homeostatic control systems.

As used herein, the term “effector molecule” refers to a molecule within a subject that can directly or indirectly regulate the metabolic activity of a target cell by increasing or decreasing the production of DNA, RNA and/or amino-acid sequences and/or by increasing or decreasing any post-translational modifications of one or more proteins.

As used herein, the term “endogenous” refers to the production and/or modification of a molecule that originates within a subject.

As used herein, the term “excipient” refers to any substance, not itself an agent, which may be used as a component within a pharmaceutical composition or a medicament for administration of a therapeutically effective amount of the agent to a subject. Additionally or alternatively, an excipient may, either alone or in combination with further chemical components, improve the handling and/or storage properties and/or permit or facilitate formation of a dose unit of the agent. Excipients include, but are not limited to, one or more of: a binder, a disintegrant, a diluent, a buffer, a taste enhancer, a solvent, a thickening agent, a gelling agent, a penetration enhancer, a solubilizing agent, a wetting agent, an antioxidant, a preservative, a surface active agent, a lubricant, an emollient, a substance that is added to mask or counteract a disagreeable odor, fragrances or taste, a substance added to improve appearance or texture of the composition and/or a substance that is used to form the pharmaceutical compositions or medicaments. Any such excipients can be used in any dosage forms according to the present disclosure. The foregoing classes of excipients are not meant to be exhaustive but are provided merely to be illustrative of what a person of skill in the art would know and would also recognize that additional types and combinations of excipients may be used to achieve delivery of a therapeutically effective amount of the agent to a subject through one or more routes of administration.

As used herein, the term “exogenous” refers to a molecule that is within a subject but that did not originate within the subject.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%, 100%, or any amount of reduction in between the specifically recited percentages, as compared to native or control levels.

As used herein, the term “medicament” refers to a medicine and/or pharmaceutical composition that comprises the agent and that can promote recovery from a disease, disorder or symptom thereof and/or that can prevent a disease, disorder or symptom thereof and/or that can inhibit the progression of a disease, disorder, or symptom thereof.

As used herein, the term “patient” refers to a subject that is afflicted with a disease or disorder. The term “patient” includes human and veterinary subjects.

As used herein, the term “pharmaceutical composition” means any composition comprising, but not necessarily limited to, an agent to be administered a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active ingredients such as antimicrobial agents, anti-inflammatory agents, anaesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the agent. Some examples of pharmaceutically acceptable carriers and their formulations are described in Remington (1995, The Science and Practice of Pharmacy (19th ed.) ed. A. R. Gennaro, Mack Publishing Company, Easton, Pa.), the disclosure of which is incorporated herein by reference. Typically, an appropriate amount of a pharmaceutically acceptable carrier is used in the formulation to render said formulation isotonic. Examples of suitable pharmaceutically acceptable carriers include, but are not limited to: saline solutions, glycerol solutions, ethanol, N-(1(2,3-dioleyloxy)propyl)-N,N,N-trimethylammonium chloride (DOTMA), dioleolphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation should suit the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevention of” and “preventing” refer to avoiding the onset or progression of a disease, disorder, or a symptom thereof.

As used herein, the terms “production”, “producing” and “produce” refer to the synthesis and/or replication of DNA, the transcription of one or more sequences of RNA, the translation of one or more amino acid sequences, the post-translational modifications of an amino acid sequence, and/or the production of one or more regulatory molecules that can influence the production and/or functionality of an effector molecule or an effector cell. For clarity, “production” is also be used herein to refer to the functionality of a regulatory molecule, unless the context reasonably indicates otherwise.

As used herein, the terms “promote”, “promotion”, and “promoting” refer to an increase in an activity, response, condition, disease process, or other biological parameter. This can include, but is not limited to, the initiation of the activity, response, condition, or disease process. This may also include, for example, a 10% increase in the activity, response, condition, or disease as compared to the native or control level. Thus, the increase in an activity, response, condition, disease, or other biological parameter can be 10%, 20%, 30%, 40%, 50%, 60%, 70%, 80%, 90%, 100%, or more, including any amount of increase in between the specifically recited percentages, as compared to native or control levels.

As used herein, the term “prophylactic administration” refers to the administration of any composition to a subject, in the absence of any symptom or indication of a disease or disorder, to prevent the occurrence and/or progression of the disease or disorder within the subject.

As used herein, the terms “signal molecule”, “signalling molecule” and “regulatory molecule” can be used interchangeably and refer to a molecule that can directly or indirectly affect the production and/or functionality of an effector molecule or effector cell. Signal molecules can be enzymes or other types of biomolecules that can act as a direct ligand on a target cell or they may influence the levels or functionality of a downstream ligand or a receptor for a ligand.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types, ex vivo preparations, and /or a sample of biological materials such as tissue and/or biological fluids.

As used herein, the term “target cell” refers to one or more cells and/or cell types that are deleteriously affected, either directly or indirectly, by a dysregulated immune system and/or a disease process.

As used herein, the term “therapeutically effective amount” refers to the amount of the agent used that is of sufficient quantity to ameliorate, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment” refers to any treatment of a disease, disorder, or symptom thereof in a subject and includes: (a) preventing the disease from occurring in a subject which may be predisposed to the disease but has not yet been diagnosed as having it; (b) inhibiting the disease, i.e., arresting its development; and (c) ameliorating the disease.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active ingredients, or combinations thereof. The amount of agent within each unit is a therapeutically effective amount.

In one embodiment of the present disclosure, the pharmaceutical compositions disclosed herein comprise an agent as described above in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%. about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The present disclosure relates to one or more agents, therapies, treatments, and methods of use of the agents and/or therapies and/or treatments for upregulating production and/or functionality of one immune checkpoint molecules. Some embodiments of the present disclosure relate to methods for making a complex between at least one particle of an agent and at least one target cell of a subject. The complex upregulates the subject's production and/or functionality of one or more precursors of a checkpoint protein (PCPP) and/or the complex upregulates the subject's production and/or functionality of one or more regulators of one or more PCPPs. Embodiments of the present disclosure can be used as a therapy or a treatment for a subject that has a condition whereby the subject's immune system is, or is likely to become, dysregulated.

In some embodiments of the present disclosure, the agent can be administered to the subject by an intravenous route, an intramuscular route, an intraperitoneal route, an intrathecal route, an intravesical route, a topical route, an intranasal route, a transmucosal route, a pulmonary route, and combinations thereof

In some embodiments of the present disclosure, the agent can be administered to the subject by pipetting a dose of the agent into an in vitro cell culture, perfusing or immersing an ex vivo cell or tissue preparation with a solution that comprises the agent, mixing a biological fluid sample with a solution or substrate that comprises the agent, or combinations thereof.

Some embodiments of the present disclosure relate to an agent that can be administered to a subject with the condition. When a therapeutically effective amount of the agent is administered to the subject, the subject may change production and/or functionality of one or more immune system molecules. For example, the subject may increase or decrease production and/or functionality of one or more immune system signaling molecules and/or one or more immune system effector molecules by changing the production of one or more sequences of DNA, one or more sequences of RNA and/or one or more proteins and/or one or more regulatory molecules that regulate the levels and/or functionality of the subject's immune system signaling molecules and/or immune system effector molecules.

In some embodiments of the present disclosure, the subject may respond to receiving the therapeutic amount of the agent by changing production and/or functionality of one or more intermediary molecules by changing production of one or more DNA sequences, one or more RNA sequences, and/or one or more proteins that regulate the levels and/or functionality of the one or more intermediary molecules. The one or more intermediary molecules regulate the subject's levels and/or functionality of the one or more immune system signaling molecules and/or the one or more immune system effector molecules.

In some embodiments of the present disclosure, administering a therapeutic amount of the agent to a subject upregulates the production, functionality or both of one or more PCPPs and the agent upregulates the production, functionality or both of one or more regulatory molecules of one or more PCPPs. The agent can upregulate production of the one or more PCPP and regulatory molecules of a PCPP by increasing one or more of: synthesis of one or more nucleotides, nucleosides, sequences or genes that are related to increased amounts or functionality of PCPP; transcription of RNA that is related to increased amounts or functionality of PCPP; or translation of one or more amino acids or amino acid sequences that are related to increased amounts or functionality of PCPP. Examples of PCPPs that the agent can upregulate the production or functionality of include, but are not limited to the precursor proteins of: cytotoxic T-lymphocyte associated protein 4 (CTLA-4), programmed cell death protein 1 (PD-1), programmed death ligand 1 (PD-L1), programmed death ligand 2 (PD-L2), indoleamine 2,3-dioxygenase 1 (IDO-1), or combinations thereof.

In some embodiments of the present disclosure, administering a therapeutic amount of the agent to a subject upregulates the production, functionality or both of one or more regulatory molecules that regulates the production or functionality of one or more PCPPs and/or one or more regulatory molecule of one or more PCPPs. The one or more regulatory molecules can be a sequence of DNA, RNA or amino acids that causes an increase in the production or functionality of one or more PCPP and regulatory molecules of a PCPP after administration of the agent. The agent can upregulate the production or functionality of the one or more regulatory molecules by increasing one or more of: synthesis of one or more nucleotides, nucleosides, sequences or genes that are related to stimulating or otherwise causing increased amounts or functionality of the one or more regulatory molecules; transcription of RNA that is related to increased amounts or functionality of the one or more regulatory molecules; or translation of one or more amino acids or amino acid sequences that are related to stimulating or otherwise causing increased amounts or functionality of the one or more regulatory molecules. Examples of such regulatory molecules are a sequence of DNA or a sequence of RNA that causes increased amounts or functionality of the precursor protein of INF-γ.

In some embodiments of the present disclosure, the agent is a vector used for gene therapy. The gene therapy is useful for increasing the production of one or more PCPP and regulatory molecules of PCPP. For example, the vector can contain a gene that causes increased expression of the precursor proteins of INF-y, CTLA-4, PD-1, PD-L1, PD-L2, IDO-1, and combinations thereof.

In some embodiments of the present disclosure, the vector used for gene therapy is a virus or recombinant virus that can be within one or more of the following genera: flavivirus, influenza virus, enterovirus, rotavirus, rubellavirus, rubivirus, morbillivirus, orthopoxvirus, varicellovirus, dependoparvovirus, alphabaculovirus, betabaculovirus, deltabaculovirus, gammabaculovirus, mastadenovirus, simplexvirus, varicellovirus, cytomegalovirus, or combinations thereof.

The embodiments of the present disclosure also relate to administering a therapeutically effective amount of the agent. In some embodiments of the present disclosure, the therapeutically effective amount of the agent that is administered to a patient is between about 10 and about 1×10¹⁶ TCID₅₀/kg (50% tissue culture infective dose per kilogram of the patient's body weight). In some embodiments of the present disclosure, the therapeutically effective amount of the agent that is administered to the patient is about 1×10¹³ TCID₅₀/kg. In some embodiments of the present disclosure, the therapeutically effective amount of the agent that is administered to a patient is measured in TPC/kg (total particle count of the agent per kilogram of the patient's body weight). In some embodiments the therapeutically effective amount of the agent is between about 10 and about 1×10¹⁶ TCP/kg.

Some embodiments of the present disclosure relate to a method for making a complex within a subject. The method comprises a step of administering a therapeutically effective amount of the agent to the subject. The complex comprises at least one particle of agent and one or more target cells. When the complex is formed, it affects a change in metabolism of the one or more target cells, which results in the subject upregulating the production and/or functionality of one or more PCPPs and/or one or regulatory molecules of one or more PCPPs. Examples of a target cell include, but are not limited to: an adrenal gland cell; a B cell; a bile duct cell; a chondrocyte; a cochlear cell; a corneal cell; an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a T cell; a testicular tissue cell; a thyroid gland cell; a uveal cell; or combinations thereof.

Some embodiments of the present disclosure relate to a therapy that can be administered to a subject with the condition. The therapy comprises a step of administering to the subject a therapeutically effective amount of an agent that will upregulate production or activity of one or more regulatory molecules and/or one or more PCPPs and/or one or more regulatory molecules of one or more PCPPs. When the therapy is administered to a patient, the therapy will promote the in vivo production and/or functionality of one or more PCPPs and/or one or more regulatory molecules of one or more PCPPs. The increased production and/or functionality of one or more PCPPs may reduce deleterious effects of the condition upon the patient.

Some embodiments of the present disclosure relate to a method of treating a condition wherein the method comprises a step of administering to the subject a therapeutically effective amount of an agent that will upregulate production or activity of one or more PCPPs and/or one or more regulatory molecules of one or more PCPPs.

Some embodiments of the present disclosure relate to one or more PCPPs of the following amino acid sequences:

```
(CTLA-4 precursor)
SEQUENCE ID 001
        10 20 30 40
MACLGFQRHK AQLNLATRTW PCTLLFFLLF IPVFCKAMHV
                50 60 70 80
AQPAVVLASS RGIASFVCEY ASPGKATEVR VTVLRQADSQ
                90 100 110 120
VTEVCAATYM MGNELTFLDD SICTGTSSGN QVNLTIQGLR
              130 140 150 160
AMDTGLYICK VELMYPPPYY LGIGNGTQIY VIDPEPCPDS
              170 180 190 200
DFLLWILAAV SSGLFFYSFL LTAVSLSKML KKRSPLTTGV
              210 220
YVKMPPTEPE CEKQFQPYFI PIN
(IDO-1 precursor)
SEQUENCE ID 002
                10 20 30 40
MAHAMENSWT ISKEYHIDEE VGFALPNPQE NLPDFYNDWM
                50 60 70 80
FIAKHLPDLI ESGQLRERVE KLNMLSIDHL TDHKSQRLAR
                90 100 110 120
LVLGCITMAY VWGKGHGDVR KVLPRNIAVP YCQLSKKLEL
              130 140 150 160
PPILVYADCV LANWKKKDPN KPLTYENMDV LFSFRDGDCS
              170 180 190 200
KGFFLVSLLV EIAAASAIKV IPTVFKAMQM QERDTLLKAL
              210 220 230 240
LEIASCLEKA LQVFHQIHDH VNPKAFFSVL RIYLSGWKGN
              250 260 270 280
PQLSDGLVYE GFWEDPKEFA GGSAGQSSVF QCFDVLLGIQ
              290 300 310 320
QTAGGGHAAQ FLQDMRRYMP PAHRNFLCSL ESNPSVREFV
              330 340 350 360
LSKGDAGLRE AYDACVKALV SLRSYHLQIV TKYILIPASQ
              370 380 390 400
QPKENKTSED PSKLEAKGTG GTDLMNFLKT VRSTTEKSLL
KEG
(PD-1 precursor)
SEQUENCE ID 003
                10 20 30 40
MQIPQAPWPV VWAVLQLGWR PGWFLDSPDR PWNPPTFSPA
                50 60 70 80
LLVVTEGDNA TFTCSFSNTS ESFVLNWYRM SPSNQTDKLA
                90 100 110 120
AFPEDRSQPG QDCRFRVTQL PNGRDFHMSV VRARRNDSGT
              130 140 150 160
YLCGAISLAP KAQIKESLRA ELRVTERRAE VPTAHPSPSP
              170 180 190 200
RPAGQFQTLV VGVVGGLLGS LVLLVWVLAV ICSRAARGTI
              210 220 230 240
GARRTGQPLK EDPSAVPVFS VDYGELDFQW REKTPEPPVP
              250 260 270 280
CVPEQTEYAT IVFPSGMGTS SPARRGSADG PRSAQPLRPE
DGHCSWPL
(PD-L1 precursor)
SEQUENCE ID 004
                10 20 30 40
MRIFAVFIFM TYWHLLNAFT VTVPKDLYVV EYGSNMTIEC
                50 60 70 80
KFPVEKQLDL AALIVYWEME DKNIIQFVHG EEDLKVQHSS
                90 100 110 120
YRQRARLLKD QLSLGNAALQ ITDVKLQDAG VYRCMISYGG
              130 140 150 160
ADYKRITVKV NAPYNKINQR ILVVDPVTSE HELTCQAEGY
              170 180 190 200
PKAEVIWTSS DHQVLSGKTT TTNSKREEKL FNVTSTLRIN
              210 220 230 240
TTTNEIFYCT FRRLDPEENH TAELVIPELP LAHPPNERTH
              250 260 270
LVILGAILLC LGVALTFIFR LRKGRMMDVK KCGIQDTNSK
          280 290
KQSDTHLEET
(PD-L2 precursor)
SEQUENCE ID 005
                10 20 30 40
MIFLLLMLSL ELQLHQIAAL FTVTVPKELY IIEHGSNVTL
                50 60 70 80
ECNFDTGSHV NLGAITASLQ KVENDTSPHR ERATLLEEQL
                90 100 110 120
PLGKASFHIP QVQVRDEGQY QCIIIYGVAW DYKYLTLKVK
              130 140 150 160
ASYRKINTHI LKVPETDEVE LTCQATGYPL AEVSWPNVSV
              170 180 190 200
PANTSHSRTP EGLYQVTSVL RLKPPPGRNF SCVFWNTHVR
              210 220 230 240
ELTLASIDLQ SQMEPRTHPT WLLHIFIPFC IIAFIFIATV
              250 260 270
IALRKQLCQK LYSSKDTTKR PVTTTKREVN SAI
(INF-γ precursor)
SEQUENCE ID 006
                10 20 30 40
MKYTSYILAF QLCIVLGSLG CYCQDPYVKE AENLKKYFNA
                50 60 70 80
GHSDVADNGT LFLGILKNWK EESDRKIMQS QIVSFYFKLF
                90 100 110 120
KNFKDDQSIQ KSVETIKEDM NVKFFNSNKK KRDDFEKLTN
              130 140 150 160
YSVTDLNVQR KAIHELIQVM AELSPAAKTG KRKRSQMLFR
GRRASQ
```

### EXAMPLE 1

In one example, the agent is a recombinant virus vector such as a AAV6.2FF gene vector that comprises a gene insert for the gene responsible for upregulating the production of an INF-γ precursor protein in humans.

In this example, the gene insert for the INF-γ precursor protein produces a biological compound from the following amino acid sequence for the INF-γ precursor protein (SEQ ID NO. 6):

MKYTSYILAFQLCIVLGSLGCYCQDPYVKEAENLKKYFNAGHSDVADNGT
LFLGILKNWKEESDRKEVIQSQIVSFYFKLFKNFKDDQSIQKSVETIKED
MNVKFFNSNKKKRDDFEKLTNYSVTDLNVQRKAIHELIQVMAELSPAAKT
GKRKRSQMLFRGRRASQ

The invention claimed is:

1. A recombinant virus vector (RVV) comprising a virus with a gene insert coding for a precursor protein of one or more of: human cytotoxic T-lymphocyte associated protein 4 (CTLA-4) (SEQ ID NO. 1), programmed cell death protein 1 (PD-1) (SEQ ID NO. 2), programmed death ligand 1 (PD-L1) (SEQ ID NO. 3), programmed death ligand 2 (PD-L2) (SEQ ID NO. 4), indoleamine 2, 3-dioxygenase 1 (IDO-1) (SEQ ID NO. 5), and gamma interferon (INF-γ) (SEQ ID NO. 6).

2. A recombinant virus vector (RVV) comprising a virus with a gene insert coding a peptide comprising ten or more amino acids in a sequence of: SEQ ID NO. 1, SEQ ID NO. 2, SEQ ID NO. 3, SEQ ID NO. 4, SEQ ID NO. 5, and SEQ ID NO. 6.

3. The RVV of claim 1 or claim 2, wherein the RVV is of a genus that is one or more of a flavivirus, an influenza virus, an enterovirus, a rotavirus, a rubellavirus, a rubivirus, a morbillivirus, an orthopoxvirus, a varicellovirus, a dependoparvovirus, an alphabaculovirus, a betabaculovirus, a deltabaculovirus, a gammabaculovirus, a mastadenovirus, a rubulavirus, a simplexvirus, a varicellovirus, a vesiculovirus, a lyssavirus, a cytomegalovirus and combinations thereof.

4. A method of making an agent/target cell complex, the method comprising a step of administering a recombinant virus vector (RVV) to a target cell for forming the agent/target cell complex, wherein the agent/target cell complex causes the target cell to increase production of a peptide sequence of one or more of: SEQ ID NO. 1, SEQ ID NO. 2, SEQ ID NO. 3, SEQ ID NO. 4, SEQ ID NO. 5, and SEQ ID NO. 6.

5. The method of claim 4, wherein the target cell is one or more of an adrenal gland cell; a B cell; a bile duct cell; a chondrocyte; a cochlear cell; a corneal cell; a dendritic cell, an endocardium cell; an endometrial cell; an endothelial cell; an epithelial cell;
an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a stem cell, a T cell; a testicular tissue cell; a thyroid gland cell; an uveal cell; and
combinations thereof.

6. A pharmaceutical agent comprising:
a. an agent that upregulates production of one or more of peptide sequences of:
SEQ ID NO. 1, SEQ ID NO. 2, SEQ ID NO. 3, SEQ ID NO. 4, SEQ ID NO. 5, and SEQ ID NO. 6;
b. a pharmaceutically acceptable carrier; and/or
c. an excipient.

7. The pharmaceutical agent of claim 6, wherein the pharmaceutical agent is in a solid form or a fluid form.

8. A method of treating a condition, the method comprising a step of administering to a subject a therapeutically effective amount of an agent for upregulating the subject's production of one or more proteins and/or peptides selected from one or more of SEQ ID NO. 1, SEQ ID NO. 2, SEQ ID NO. 3, SEQ ID NO. 4, SEQ ID NO. 5, and SEQ ID NO. 6.

9. The method of claim 8, further comprising a step of administering at least one of radiotherapy, chemotherapy and a biological-based chemotherapy agent.

10. The method according to claim 8, wherein the step of administering the agent occurs by an intravenous route, an intramuscular route, an intraocular route, an intraperitoneal route, an intrathecal route, an intravesical route, a topical route, an intranasal route, a transmucosal route, a pulmonary route, and combinations thereof.

11. The method according to claim 8, wherein the therapeutically effective amount is between about 10 to about 1×10¹⁶ TCID₅₀/kg of the patient's body weight.

12. The method according to claim 8, wherein the therapeutically effective amount is between about 10 to about 1×10¹⁶ total particles/kg of the agent.

13. The method according to claim 8, wherein the therapeutically effective amount is between about 10 to about 1×10¹⁶ VG/kg of the agent.


SEQUENCE LISTING (6 sequences)

SEQ ID NO: 1 - protein, 223 aa, Artificial Sequence
  Synthetic Sequence
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 2 - protein, 403 aa, Artificial Sequence [sequence omitted]

SEQ ID NO: 3 - protein, 288 aa, Artificial Sequence
  Synthetic Sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - protein, 290 aa, Artificial Sequence
  Synthetic Sequence
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 5 - protein, 273 aa, Artificial Sequence
  Synthetic Sequence
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 6 - protein, 166 aa, Artificial Sequence
  Synthetic Sequence
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ